(19) United States
(12) Patent Application Publication
(10) Pub. No.: US 2023/0064955 A1
(43) Pub. Date: Mar. 2, 2023

(54) SYSTEMS AND METHODS FOR MEDICAL PATIENT DISTRACTION

(71) Applicant: Matthew Parker, McKinney, TX (US)

(72) Inventor: Matthew Parker, McKinney, TX (US)

(21) Appl. No.: 17/890,075

(22) Filed: Aug. 17, 2022

Related U.S. Application Data
(60) Provisional application No. 63/236,719, filed on Aug. 25, 2021.

(51) Int. Cl. A61M 21/02; H04N 5/64; H04R 1/10; G10K 11/178; A61C 19/08; A61M 16/10; A61M 16/06

(52) U.S. Cl. CPC A61M 21/02; H04N 5/642; H04R 1/1083; H04R 1/1008; G10K 11/178; A61C 19/08; A61M 16/104; A61M 16/06; G06F 3/1454

(57) ABSTRACT

Systems and methods for medical patient distraction may permit watching an immersive video headset during a dental or medical procedure that is compatible with nitrous oxide. Having the ability to watch a show on an immersive video headset while using nitrous oxide may allow for further distraction of the patient from the discomfort of dental or medical procedures. The parts and pieces of the immersive video headset that touch the patient may be detachable so they can be changed between patients for easy sterilization and cleanliness. Also, the immersive video headset may be capable of mirroring what is on a phone or nearby computer. The design of the headset may be specifically compatible with being able to use a nitrous hood during dental or medical procedures.

### CROSS-REFERENCE TO RELATED APPLICATION

The present application is a non-provisional of, and claims priority to, U.S. Patent Application No. 63/236,719 filed Aug. 25, 2021, the disclosure of which is incorporated by reference in its entirety.

### FIELD OF THE DISCLOSURE

The present disclosure generally relates systems and methods for medical patient distraction, and more particularly to systems and methods for distraction of a medical patient during a procedure with a cinematic personal theatre (immersive video headset) and nitrous oxide.

### BACKGROUND

It is desirable to distract medical patients during procedures using audio and/or video headsets. However, there are no video headsets that are compatible with nitrous hood used during routine dental and medical procedures. Nitrous now is offered for patients during the labor and delivery process. The delivery of nitrous oxide is closely regulated and has specific nose pieces that cannot be modified for this type of device due to medical board regulations and patient safety. The patient must choose either video or the nitrous hood. Accordingly, the patient is either distracted through video or made less painful through use of the nitrous hood, and this can result in a procedure that is not very comfortable for the patient. In addition, available video headsets have various problems. They get in the way or are too bulky, they do not have sound capability, there are cords that get in the way when you are trying to perform dental or medical procedures, they are not noise canceling, they do not utilize Bluetooth, they are not medical grade or durable for commercial use, and/or they cannot be wiped down without destroying them.

### SUMMARY

Embodiments of the present disclosure may provide systems and methods for medical patient distraction that may permit watching an immersive video headset during a dental or medical procedure that is compatible with nitrous oxide. Systems and methods according to embodiments of the present disclosure could be used across many other medical subspecialties and disciplines for procedural comforting. Having the ability to watch a show on an immersive video headset while using nitrous oxide may allow for further distraction of the patient from the discomfort of dental or medical procedures. In embodiments of the present disclosure, the parts and pieces of the immersive video headset that touch the patient may be detachable so they can be changed between patients for easy sterilization and cleanliness. In some embodiments, replacement pieces may be manufactured to be durable and able to withstand the harsh chemicals required for cleaning and sterilizing to allow the parts that touch the patient and are most frequently subjected to harsh chemicals for cleaning to be replaceable without replacing the entire headset. Also, the immersive video headset may be capable of mirroring what is on a phone or nearby computer. The design of the headset may be specifically compatible with being able to use a nitrous hood during dental or medical procedures.

Embodiments of the present disclosure may provide a system for medical patient distraction comprising: an immersive video headset providing a cinematic personal theatre for a medical patient; and a nitrous hood having a nitrous nose piece to deliver nitrous oxide to the medical patient, wherein the immersive video headset may be compatible with simultaneous use of the nitrous hood. The immersive video headset may have a plurality of disposable pads that may be used on parts of the immersive video headset that touch or contact with the medical patient. The immersive video headset may have a plurality of snap-on parts that touch or contact with the medical patient, wherein the plurality of snap-on parts can be detached for sterilization. The immersive video headset may be formed of durable, medical grade material to withstand use of anti-viral and/or anti-bacterial wipes. The immersive video headset also may include anti-fogging lenses. The immersive video headset may allow for adjustment of interpupillary distance and focusing for the medical patient having glasses or less than perfect vision. The immersive video headset may include screen mirroring so that medical personnel can view what the medical patient is watching and make adjustments. The immersive video headset may be wireless. The nitrous nose piece may be disposable. The nitrous nose piece may be sterilized between medical patients.

Other embodiments of the present disclosure may provide a system for medical patient distraction comprising: an immersive video headset providing a cinematic personal theatre for a medical patient, the immersive video headset having noise canceling headphone portions with snap-on disposable pads; and a nitrous hood having a nitrous nose piece to deliver nitrous oxide to the medical patient, wherein the noise canceling headphone portions may be disposed on each side of the nitrous nose piece, wherein the immersive video headset may be compatible with simultaneous use of the nitrous hood. The snap-on disposable pads may include a removable head pad; and removable eye pads. The immersive video headset may include a virtual reality (VR) goggle positioned above the nitrous nose piece; and arms that are connected to and operational with the noise canceling headphone portions and the VR goggle. The nitrous nose piece may be adjustable. The immersive video headset may include anti-fogging lenses, and they may be rotatable to focus and adjustable side-to-side. The immersive video headset may allow for adjustment of interpupillary distance and focusing for the medical patient having glasses or less than perfect vision.

Further embodiments of the present disclosure may provide a system for medical patient distraction comprising: a nitrous hood having a nitrous nose piece to deliver nitrous oxide to the medical patient; and an immersive video headset providing a cinematic personal theatre for a medical patient, the immersive video headset having a disposable head pad and disposable eye pads, and a virtual reality (VR) goggle positioned above the nitrous nose piece, wherein the immersive video headset may be compatible with simultaneous use of the nitrous hood. The disposable head pad and disposable eye pads may be snap-on. The immersive video headset also may include screen mirroring so that medical personnel can view what the medical patient is watching and make adjustments.

### BRIEF DESCRIPTION OF THE DRAWINGS

For a more complete understanding of this disclosure, reference is now made to the following description, taken in conjunction with the accompanying drawings, in which:

FIG. 1 depicts a front view of an immersive video headset according to an embodiment of the present disclosure;

FIG. 2 depicts a side view of an immersive video headset according to an embodiment of the present disclosure;

FIG. 3 depicts a back view of an immersive video headset according to an embodiment of the present disclosure; and

FIG. 4 depicts a bottom view of an immersive video headset according to an embodiment of the present disclosure.

### DETAILED DESCRIPTION

Embodiments of the present disclosure may provide systems and methods for medical patient distraction providing a cinematic personal theatre utilizing an immersive video headset that is compatible with simultaneous use of a nitrous hood. The immersive video headset may provide for easy sterilization and cleanliness while allowing the patient to utilize a nitrous hood during the procedure as desired.

Visualization of the operator/clinician is paramount. The design must be specific for the operator/clinician to be able to see and for the patient to be able to enjoy the experience. Accordingly, the design should maximize the optics for the patient and maximize visualization for the operator/clinician. The immersive video headset according to embodiments of the present disclosure may be easy to clean between patients. In an embodiment of the present disclosure, the parts of the immersive video headset that touch or contact with the patient may be formed of disposable foam, such as disposable pads, that may be easily changed out between patients. In some embodiments of the present disclosure, the immersive video headset may include parts that can attach and snap for sterilization purposes. The immersive video headset also may be sufficiently durable and medical grade to withstand use of various chemicals that may be utilized to wipe the headset down with anti-viral and/or bacterial wipes.

The immersive video headset also may include anti-fogging lenses so that when the headset is first placed on the patient, the lenses are not blurry even as the temperature increases around the lenses when placed in close proximity to body heat. The immersive video headset also may allow for adjustment of interpupillary distance and focusing for people who have glasses and or less than perfect vision.

The immersive video headset according to embodiments of the present disclosure may include screen mirroring capability so that medical personnel, such as the doctor, nurse, or assistant, can see what the patient is watching, allowing them to change the show, or make adjustments during the procedure. Screen mirroring occurs by turning one device into the sender and the other the receiver so that whatever action the patient does on the immersive video headset may appear on another screen in real time. This may be performed through a wired mechanism, such as through use of an HDMI cable to connect the immersive video headset to an external display. Additionally, or alternatively, a wireless mechanism, such as Apple TV, Google Chromecast, or Microsoft Wireless Display Adapter, may be used as a wireless screen mirroring device to display content.

The immersive video headset may be wireless so that it may be less bulky with fewer wires to get in the way during a medical procedure. The immersive video headset according to embodiments of the present disclosure may include more than one battery pack that can be interchanged and charged throughout the day so that each patient may utilize the headset without much, if any, downtime between patients.

The immersive video headset according to embodiments of the present disclosure may be compatible for use of a nitrous hood to deliver nitrous oxide to a patient. A nitrous nose piece may be provided as a modified disposable component in some embodiments of the present disclosure. However, there may be other embodiments of the present disclosure where the immersive video headset may be designed around the standard nitrous nose pieces that may be provided for use with a nitrous hood. Various sizes (typically, small, medium, and large) may be provided, and the standard nitrous nose piece may be reusable and sterilized between patients in some embodiments of the present disclosure. Other components of the immersive video headset may be formed of a standard medical grade plastic in embodiments of the present disclosure.

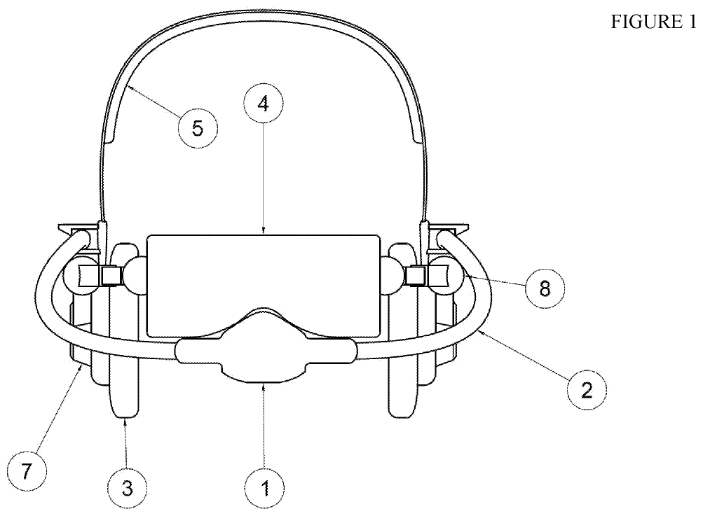

FIG. 1 depicts a front view of an immersive video headset according to an embodiment of the present disclosure. As depicted herein, noise canceling headphone portions 3 of the headset may be disposed on either side of the nitrous nose piece or nasal hood 1. Tubes 2 may extend from each side of nasal hood 1 above right arm 7 and left arm 8 respectively. The headset also may include snap-on disposable pads 5, 6 for easy cleaning between patients. More specifically, the disposable pads may include removable head pad 5 and removable eye pads (not shown).

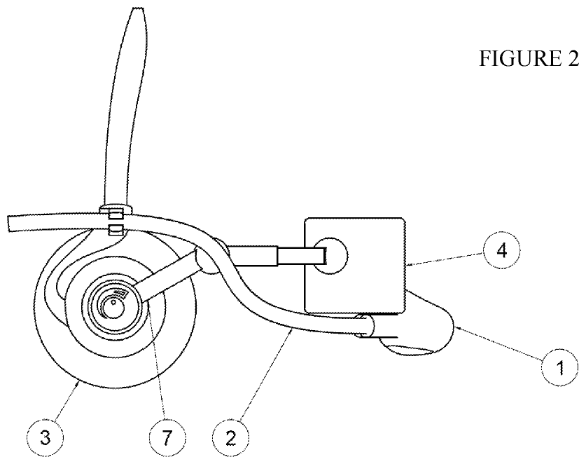

FIG. 2 depicts a side view of an immersive video headset according to an embodiment of the present disclosure. As described with respect to FIG. 1, one or more snap-on disposable pads may be changed out between patients. The nitrous nose piece or nasal hood 1 may be adjustable in embodiments of the present disclosure. Virtual reality (VR) goggle 4 may be positioned above nasal hood 1 so as to not interfere with operation of nasal hood 1 relative to tubes 2. Arms 7 may be connected to and operational with headphones 3, and each of arms 7 may be connected to and in communication with VR goggle 4. Disposable foam pads may be provided around the headphones 3 that may be changed out between patients, and the headphones 3 may be adjustable to accommodate different sizes of patients.

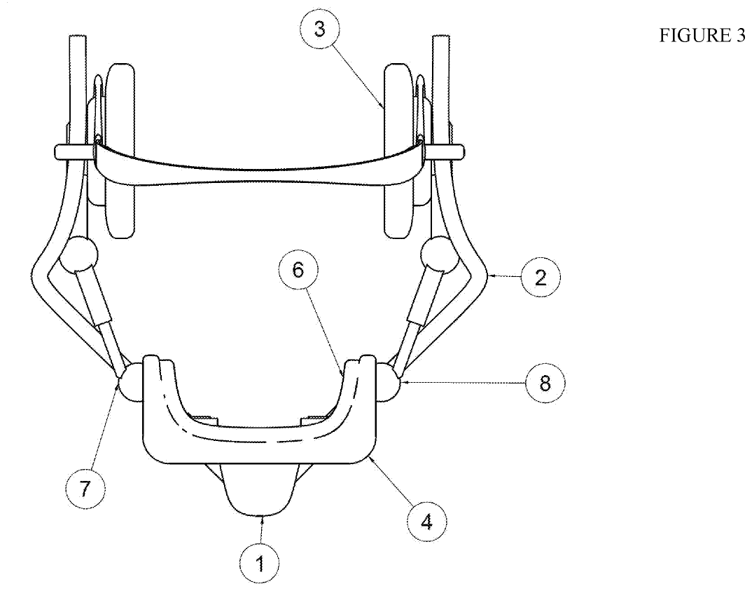

FIG. 3 depicts a back view of an immersive video headset according to an embodiment of the present disclosure. In this back view, the headphones 3 are again depicted, and they may be noise-canceling headphones in embodiments of the present disclosure. The disposable pads around the eye portions 6, forehead portion 5, and/or the headphone/ear portions of the headset also are depicted. While disposable and/or removable pads are depicted as being positioned in each of these areas in FIG. 3, there may be embodiments of the present disclosure where disposable pads may not be utilized in each of the areas. There also may be embodiments of the present disclosure where disposable pads may be positioned/utilized in more areas than specifically identified herein. It should be appreciated that the lenses of the headset may be no fog lenses in embodiments of the present disclosure. There may be an adjustable interpupillary distance between the lenses in embodiments of the present disclosure. The nitrous nasal hood 1 positioned beneath the lenses of the headset also is depicted in FIG. 3.

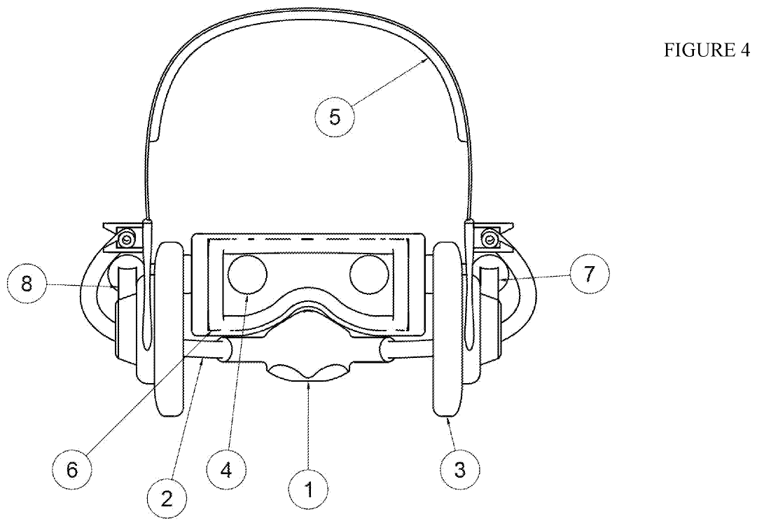

FIG. 4 depicts a bottom view of an immersive video headset according to an embodiment of the present disclosure. As depicted herein, disposable pads are depicted at or around the eye portions and/or the headphone/ear portions of the headset. Again, as discussed with respect to FIG. 3, while disposable pads are depicted as being positioned in each of these areas, there may be embodiments of the present disclosure where disposable pads may not be utilized in each of the areas. There also may be embodiments of the present disclosure where disposable pads may be positioned/utilized in more areas than specifically identified herein. There may be an adjustable interpupillary distance between the lenses in embodiments of the present disclosure. The lenses may be rotatable to focus, and adjustments may be made side-to-side in embodiments of the present disclosure.

Although the present disclosure and its advantages have been described in detail, it should be understood that various changes, substitutions and alterations can be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A system for medical patient distraction comprising:
an immersive video headset providing a cinematic personal theatre for a medical patient; and
a nitrous hood having a nitrous nose piece to deliver nitrous oxide to the medical patient,
wherein the immersive video headset is compatible with simultaneous use of the nitrous hood.

2. The system of claim 1, the immersive video headset having a plurality of disposable pads that are used on parts of the immersive video headset that touch or contact with the medical patient.

3. The system of claim 1, the immersive video headset having a plurality of snap-on parts that touch or contact with the medical patient, wherein the plurality of snap-on parts can be detached for sterilization.

4. The system of claim 1, wherein the immersive video headset is formed of durable, medical grade material to withstand use of anti-viral and/or anti-bacterial wipes.

5. The system of claim 1, the immersive video headset further comprising:
anti-fogging lenses.

6. The system of claim 1, wherein the immersive video headset allows for adjustment of interpupillary distance and focusing for the medical patient having glasses or less than perfect vision.

7. The system of claim 1, the immersive video headset further comprising:
screen mirroring so that medical personnel can view what the medical patient is watching and make adjustments.

8. The system of claim 1, wherein the immersive video headset is wireless.

9. The system of claim 1, wherein the nitrous nose piece is disposable.

10. The system of claim 1, wherein the nitrous nose piece is sterilized between medical patients.

11. A system for medical patient distraction comprising:
an immersive video headset providing a cinematic personal theatre for a medical patient, the immersive video headset having noise canceling headphone portions with snap-on disposable pads; and
a nitrous hood having a nitrous nose piece to deliver nitrous oxide to the medical patient, wherein the noise canceling headphone portions are disposed on each side of the nitrous nose piece,
wherein the immersive video headset is compatible with simultaneous use of the nitrous hood.

12. The system of claim 11, the snap-on disposable pads comprising:
a removable head pad; and
removable eye pads.

13. The system of claim 11, the immersive video headset further comprising:
a virtual reality (VR) goggle positioned above the nitrous nose piece; and
arms that are connected to and operational with the noise canceling headphone portions and the VR goggle.

14. The system of claim 11, wherein the nitrous nose piece is adjustable.

15. The system of claim 11 the immersive video headset further comprising:
anti-fogging lenses.

16. The system of claim 16, wherein the anti-fogging lenses are rotatable to focus and adjustable side-to-side.

17. The system of claim 11, wherein the immersive video headset allows for adjustment of interpupillary distance and focusing for the medical patient having glasses or less than perfect vision.

18. A system for medical patient distraction comprising:
a nitrous hood having a nitrous nose piece to deliver nitrous oxide to the medical patient; and
an immersive video headset providing a cinematic personal theatre for a medical patient, the immersive video headset having a disposable head pad and disposable eye pads, and a virtual reality (VR) goggle positioned above the nitrous nose piece,
wherein the immersive video headset is compatible with simultaneous use of the nitrous hood.

19. The system of claim 18, wherein the disposable head pad and disposable eye pads are snap-on.

20. The system of claim 18, the immersive video headset further comprising:
screen mirroring so that medical personnel can view what the medical patient is watching and make adjustments.
